(19) United States
(12) Patent Application Publication
(10) Pub. No.: US 2004/0235167 A1
(43) Pub. Date: Nov. 25, 2004

(54) Cell culture base material and method of producing the same

(76) Inventors: Hideyuki Miyake, Tokyo-to (JP); Hideshi Hattori, Tokyo-to (JP)

Correspondence Address: LADAS & PARRY LLP, 224 SOUTH MICHIGAN AVENUE, SUITE 1200, CHICAGO, IL, 60604, US

(21) Appl. No.: 10/847,160

(22) Filed: May 17, 2004

(30) Foreign Application Priority Data
May 20, 2003 (JP) 2003-142557

(51) Int. Cl. C12N 5/02

(52) U.S. Cl. 435/402

(57) ABSTRACT

The main object of the present invention is to provide a cell culture base material having a base material and a pattern of regions of excellent cell adhesiveness formed on the base material with high precision, and a method of producing the same.

The present invention attains the object by providing a cell culture base material comprising a base material and a cell adhesiveness changing layer formed on the base material and containing a cell adhesiveness changing material changing of adhesiveness of cells by the action of a photocatalyst in irradiation with energy, wherein on the cell adhesiveness changing layer, a cell adhesiveness changing pattern changed adhesiveness of cells is formed.

### BACKGROUND OF THE INVENTION

1. Field of the Invention

The present invention relates to a cell culture base material used for forming a cell pattern and a method of producing the same.

2. Description of the Related Art

Cell culture of various animal and plant are conducted and novel cell culture methods are developed, now. Technologies of cell culture are utilized for the purpose of clarifying the biochemical phenomena and natures of cells and producing useful substances. Further, there are trials on checking of the physiological activity and toxicity of drugs artificially synthesized using cultured cells.

Some cells, particularly a lot of animal cells have adhesion dependency of adhering to some material and growing thereon, and cannot survive for a long period of under flotation condition out of organisms. For culturing of cells having such adhesion dependency, a carrier for adhesion of cells is necessary, and in general, a culturing plate made of plastic uniformly applied with cell adhesive proteins such as collagen, fibronectin and the like. It is known that these cell adhesive proteins act on cultured cells, make adhesion of cells easy, and exert an influence on the form of cells.

On the other hand, there is reported a technology of adhering cultured cells only onto a small part on a base material and arranging them. By such a technology, it is made possible to apply cultured cells to artificial organs, biosensors, bioreactors and the like. As the method of arranging cultured cells, there is adopted a method in which a base material having a surface forming a pattern different easiness of adhesion for cells is used, cells are cultured on the surface of this base material and cells are allowed to adhere only to surfaces processed to adhere the cells, to arrange the cells.

For example, in Japanese Patent Application Laid-Open No. 2-245181, a charge-retaining medium formed an electrostatic pattern is applied to cell culture for the purpose of proliferating nerve cells in the form of circuit, and the like. Japanese Patent Application Laid-Open No. 3-7576 tries the arrangement of cell culture on the surface on which a cell non-adhesive or cell adhesive photosensitive hydrophilic polymer has been patterned by a photolithography method.

Further, Japanese Patent Application Laid-Open No. 5-176753 discloses a cell culture base material on which a substance such as collagen and the like imparting an influence on the adhesion ratio and form of cells has been patterned, and a method of producing this base material by a photolithography method. By culturing cells on such a base, a larger amount of cells are adhered on a surface on which collagen or the like has been patterned, to realize patterning of cells.

However, such patterning of cell culture regions may be required to be highly precise depending on applications. In the case of conducting patterning by a photolithography method using a photosensitive material as described above, a highly precise pattern can be obtained, however, a cell adhesive material is required to have photosensitivity, and for example, it is difficult in many cases to conduct chemical modification to impart such photosensitivity to biopolymers and the like, leading to extremely narrow width of selectivity of cell adhesive materials, problematically. In a photolithography method using a photo resist, it is necessary to use a liquid developer and the like, and these exert a negative influence in culturing cells in some cases.

Further, as the method of forming a highly precise pattern of a cell adhesive material, a Micro Contact Printing method is suggested by George M. White sides, Harvard University (for example, U.S. Pat. No. 5,512,131, U.S. Pat. No. 5,900,160, Japanese Patent Application Laid-Open No. 9-240125, Japanese Patent Application Laid-Open No. 10-12545 and the like). However, there is a problem that it is difficult to industrially produce a cell culture base material having a pattern of a cell adhesiveness material using this method.

### SUMMARY OF THE INVENTION

The present invention has been made to solve the problems and the main object thereof is to provide a cell culture base material formed a pattern of regions of excellent cell adhesiveness highly-precisely on a base material, and a method of producing the same.

The present invention provides, for solving the problems, a cell culture base material comprising a base material and a cell adhesiveness changing layer formed on the base material and containing a cell adhesiveness changing material changing of adhesiveness of cells by the action of a photocatalyst in irradiation with energy, wherein on the cell adhesiveness changing layer, a cell adhesiveness changing pattern changed adhesiveness of cells is formed. According to the present invention, a cell adhesiveness changing layer containing a cell adhesiveness changing material changing of adhesiveness of cells by the action of a photocatalyst in irradiation with energy is present, resultantly, by irradiation with energy in the form of pattern, there can be formed a cell adhesiveness changing pattern which is a highly precise pattern composed of regions of excellent cell adhesiveness and regions of poor cell adhesiveness easily on the surface of a cell adhesiveness changing layer.

The present invention includes three embodiments of a case in which the cell adhesiveness changing layer is a photocatalyst-containing cell adhesiveness changing layer containing a photocatalyst and the cell adhesiveness changing material, a case in which the cell adhesiveness changing layer has a photocatalyst treating layer containing a photocatalyst and a cell adhesiveness changing material layer formed on the photocatalyst treating layer and containing the cell adhesiveness changing material, further a case in which the cell adhesiveness changing layer is a cell adhesiveness changing material layer containing the cell adhesiveness changing material, and the cell adhesiveness changing pattern is formed by placing the photocatalyst-containing layer containing a photocatalyst and the cell adhesiveness changing material layer so that they face and then irradiating energy from given direction.

Further, in the present invention, the cell adhesiveness changing pattern may also be a cell adhesiveness changing pattern having regions differing in surface cell adhesiveness by at least 3 levels or more. The reason for this is that by use of a pattern including regions of excellent cell adhesiveness and regions of poor cell adhesiveness differing by 3 levels or more, pattern formation is possible by forming a detailed adhesiveness change pattern of multi-level or continuously changed level when what level of adhesiveness for cells is preferable is unclear, and the like.

In the present invention, “three levels or more” is a concept involving also a case of continuous change.

Further, the present invention provides a method of producing a cell culture base material comprising

a pattern forming body base material forming process of forming a pattern forming body base material having a base material and a photocatalyst-containing cell adhesiveness changing layer formed on the base material and containing a photocatalyst and a cell adhesiveness changing material changing of adhesiveness of cells by the action of the photocatalyst in irradiation with energy, and

a cell adhesiveness changing pattern forming process of irradiating the photocatalyst-containing cell adhesiveness changing layer with energy to form a cell adhesiveness changing pattern changed adhesiveness of cells of the photocatalyst-containing cell adhesiveness changing layer.

According to the present invention, because of formation of the photocatalyst-containing cell adhesiveness changing layer, a cell adhesiveness changing pattern which is a highly precise pattern composed of regions of excellent cell adhesiveness and regions of poor cell adhesiveness can be formed easily on the surface of the photocatalyst-containing cell adhesiveness changing layer, by the action of a photocatalyst contained in the photocatalyst-containing cell adhesiveness changing layer itself, by irradiation with energy.

Further, the present invention provides a method of producing a cell culture base material comprising

a pattern forming body base material forming process of forming a pattern forming body base material having a base material, a photocatalyst treating layer containing a photocatalyst formed on the base material and a cell adhesiveness changing material layer formed on the photocatalyst treating layer and containing a cell adhesiveness changing material changing of adhesiveness of cells by the action of the photocatalyst in irradiation with energy, and

a cell adhesiveness changing pattern forming process of irradiating the cell adhesiveness changing material layer with energy to form a cell adhesiveness changing pattern changed adhesiveness of cells of the cell adhesiveness changing material layer.

According to the present invention, because of formation of the photocatalyst treating layer and cell adhesiveness changing material layer, a cell adhesiveness changing pattern which is a highly precise pattern composed of regions of excellent cell adhesiveness and regions of poor cell adhesiveness can be formed easily on the surface of the cell adhesiveness changing material layer, by the action of a photocatalyst contained in the adjacent photocatalyst treating layer, by irradiation with energy.

Furthermore, the present invention provides a method of producing a cell culture base material comprising

a pattern forming body base material forming process of forming a pattern forming body base material having a base material, and a cell adhesiveness changing material layer formed on the base material and containing a cell adhesiveness changing material changing of adhesiveness of cells by the action of a photocatalyst in irradiation with energy, and

a cell adhesiveness changing pattern forming process of placing the pattern forming body base material and a photocatalyst-containing layer-side base plate having a substrate and a photocatalyst-containing layer containing a photocatalyst so that the cell adhesiveness changing material layer and the photocatalyst-containing layer face, then, irradiating with energy from given direction to form a cell adhesiveness changing pattern changed adhesiveness of cells of the cell adhesiveness changing material layer.

According to the present invention, a cell adhesiveness changing pattern which is a highly precise pattern composed of regions of excellent cell adhesiveness and regions of poor cell adhesiveness can be formed easily on the surface of the cell adhesiveness changing material layer, by irradiating the cell adhesiveness changing material layer with energy using the photocatalyst-containing layer-side base plate.

### BRIEF DESCRIPTION OF THE DRAWINGS

FIG. 1 is a process view showing one example of the method of producing a cell culture base material of the present invention.

FIG. 2 is a process view showing another example of the method of producing a cell culture base material of the present invention.

FIG. 3 is a process view showing another example of the method of producing a cell culture base material of the present invention.

FIG. 4 is a schematic sectional view showing one example of the photocatalyst-containing layer-side base plate in the present invention.

FIG. 5 is a schematic sectional view showing another example of the photocatalyst-containing layer-side base plate in the present invention.

FIG. 6 is a schematic sectional view showing another example of the photocatalyst-containing layer-side base plate in the present invention.

### DESCRIPTION OF THE PREFERRED EMBODIMENTS

The present invention relates to a cell culture base material used for forming a cell pattern, and a method of producing the same, and these will be illustrated below.

### I. Cell Culture Base Material

First, the cell culture base material of the present invention will be described in detail. The cell culture base material of the present invention comprises a base material and a cell adhesiveness changing layer formed on the base material and containing a cell adhesiveness changing material changing of adhesiveness of cells by the action of a photocatalyst in irradiation with energy, wherein on the cell adhesiveness changing layer, a cell adhesiveness changing pattern changed adhesiveness of cells is formed.

The cell culture base material of the present invention has a cell adhesiveness changing layer containing a cell adhesiveness changing material as described above, consequently, by irradiation with energy in the form of pattern along a pattern required, a pattern of changed cell adhesiveness can be formed easily on the cell adhesiveness changing layer. Therefore, a highly precise pattern can be formed by a simple process without using treating liquid exerting a negative influence on cells. Because of no necessity of modification of a cell adhesiveness changing material, width of selection of materials can be enlarged, and also a biological cell adhesiveness changing material manifesting specific adhesiveness as described later can be used without problems.

The cell culture base material of the present invention contains at least three embodiments. These will be explained individually.

A. First Embodiment

The first embodiment of the cell culture base material of the present invention is a cell culture base material comprising a base material and a cell adhesiveness changing layer formed on the base material and containing a cell adhesiveness changing material changing of adhesiveness of cells by the action of a photocatalyst in irradiation with energy, wherein on the cell adhesiveness changing layer, a cell adhesiveness changing pattern changed adhesiveness of cells is formed, being characterized in that the cell adhesiveness changing layer is a photocatalyst-containing cell adhesiveness changing layer containing a photocatalyst and the cell adhesiveness changing material.

In this embodiment, the cell adhesiveness changing layer is a photocatalyst-containing cell adhesiveness changing layer containing a photocatalyst and the cell adhesiveness changing material as described above, therefore, in irradiating with energy, the cell adhesiveness of the cell adhesiveness changing material changes by the action of the photocatalyst in the photocatalyst-containing cell adhesiveness changing layer, consequently, a cell adhesiveness changing pattern can be formed differing in cell adhesiveness between parts irradiated with energy and parts not irradiated.

The cell culture base material of this embodiment as described above will be divided into members used and illustrated respectively.

1. Photocatalyst-containing Cell Adhesiveness Changing Layer

This embodiment is characterized in that a photocatalyst-containing cell adhesiveness changing layer is formed on a base material. This photocatalyst-containing cell adhesiveness changing layer has at least a photocatalyst and a cell adhesiveness changing material.

(1) Cell Adhesiveness Changing Material

The cell adhesiveness changing material used in this embodiment is not particularly restricted providing the adhesiveness of cells changes by the action of a photocatalyst in irradiation with energy, and includes mainly two embodiments, depending on embodiments of controlling adhesiveness with cells, of a physicochemical cell adhesiveness changing material adhering to cells by a physicochemical property and a biological cell adhesiveness changing material adhering to cells by a biological property.

a. Physicochemical Cell Adhesiveness Changing Material

As the physicochemical factor for adhering cells to a base material and culturing cells, and examples thereof include a factor regarding surface free energy, a factor by hydrophobic interaction and the like, and other factors.

The physicochemical cell adhesive material having physicochemical cell adhesiveness by such factors is preferably a material having high bonding energy so that the main skeleton is not decomposed by the action of a photocatalyst and having an organic substituent decomposed by the action of a photocatalyst, and examples thereof include, (1) organo polysiloxanes causing hydrolysis and polycondensation of chloro or alkoxysilanes and the like by a sol-gel reaction and the like to manifest large strength, (2) organo polysiloxanes obtained by cross-linking of reactive silicone, and the like.

In the case (1), preferable are organo polysiloxanes which are a hydrolysis condensate or co-hydrolysis condensate of one or two silicon compounds of the general formula:

YₙSiX₍₄₋ₙ₎

(wherein, Y represents an alkyl group, fluoroalkyl group, vinyl group, amino group, phenyl group or epoxy group, X represents an alkoxyl group, acetyl group or halogen. n represents an integer of 0 to 3.).

Here, the number of carbon atoms of the group represented by Y is preferably in the range from 1 to 20, and the alkoxy group represented by X is preferably a methoxy group, ethoxy group, propoxy group or butoxy group.

As the organic group, particularly polysiloxanes containing a fluoroalkyl group can be preferably used, and specific examples thereof include hydrolysis condensates and co-hydrolysis condensates of one or more of the following fluoroalkylsilanes, and those generally known as a fluorine-based silane coupling agents can be used.

CF₃(CF₂)₃CH₂CH₂Si(OCH₃)₃;

CF₃(CF₂)₅CH₂CH₂Si(OCH₃)₃;

CF₃(CF₂)₇CH₂CH₂Si(OCH₃)₃;

CF₃(CF₂)₉CH₂CH₂Si(OCH₃)₃;

(CF₃)₂CF(CF₂)₄CH₂CH₂Si(OCH₃)₃;

(CF₃)₂CF(CF₂)₆CH₂CH₂Si(OCH₃)₃;

(CF₃)₂CF(CF₂)₈CH₂CH₂Si(OCH₃)₃;

CF₃(C₆H₄)C₂H₄Si(OCH₃)₃;

CF₃(CF₂)₃(C₆H₄)C₂H₄Si(OCH₃)₃;

CF₃(CF₂)₅(C₆H₄)C₂H₄Si(OCH₃)₃;

CF₃(CF₂)₇(C₆H₄)C₂H₄Si(OCH₃)₃;

CF₃(CF₂)₃CH₂CH₂SiCH₃(OCH₃)₂;

CF₃(CF₂)₅CH₂CH₂SiCH₃(OCH₃)₂;

CF₃(CF₂)₇CH₂CH₂SiCH₃(OCH₃)₂;

CF₃(CF₂)₉CH₂CH₂SiCH₃(OCH₃)₂;

(CF₃)₂CF(CF₂)₄CH₂CH₂SiCH₃(OCH₃)₂;

(CF₃)₂CF(CF₂)₆CH₂CH₂SiCH₃(OCH₃)₂;

(CF₃)₂CF(CF₂)₈CH₂CH₂SiCH₃(OCH₃)₂;

CF₃(C₆H₄)C₂H₄SiCH₃(OCH₃)₂;

CF₃(CF₂)₃(C₆H₄)C₂H₄SiCH₃(OCH₃)₂;

CF₃(CF₂)₅(C₆H₄)C₂H₄SiCH₃(OCH₃)₂;

CF₃(CF₂)₇(C₆H₄)C₂H₄SiCH₃(OCH₃)₂;

CF₃(CF₂)₃CH₂CH₂Si(OCH₂CH₃)₃;

CF₃(CF₂)₅CH₂CH₂Si(OCH₂CH₃)₃;

CF₃(CF₂)₇CH₂CH₂Si(OCH₂CH₃)₃;

CF₃(CF₂)₉CH₂CH₂Si(OCH₂CH₃)₃;

CF₃(CF₂)₇SO₂N(C₂H₅)C₂H₄CH₂Si(OCH₃)₃

By using a polysiloxane having a fluoroalkyl group as described above as the physicochemical cell adhesive material, a part not irradiated with energy of a photocatalyst-containing cell adhesiveness changing layer can be a part having fluorine on the surface, and on a part irradiated with energy, fluorine and the like are removed, and it can be a part having an OH group and the like on the surface. Therefore, a material can be made significantly differing in surface free energy between a part irradiated with energy and a part not irradiated with energy, to increase a difference in cell adhesiveness.

As the reactive silicone (2), mentioned are compounds having a skeleton of the following general formula.

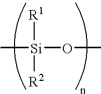

Wherein, n is an integer of 2 or more, and R¹ and R² represent each a substituted or unsubstituted alkyl, alkenyl, aryl or cyanoalkyl group having 1 to 10 carbon atoms, and 40% or less in terms of molar ratio based on the total amount are a vinyl, phenyl or halogenated phenyl. Those in which R¹ and R² represent a methyl group are preferable since the surface energy is the lowest, and those in which the molar ratio of a methyl group is 60% or more are preferable. At the chain end or side chain, at least one reactive group such as a hydroxyl group and the like is present in the molecular chain.

A stable organosilicon compound not causing a cross-linking reaction such as dimethyl polysiloxane may be separately mixed, together with the organo polysiloxanes.

On the other hand, mentioned as the physicochemical cell adhesive material of decomposition substance type are surfactants having a function of being decomposed by the action of a photocatalyst and changing polarity on the surface of a photocatalyst-containing polarity changing layer by the decomposition. Specifically listed are hydrocarbon-based surfactants such as NIKKOL BL, BC, BO and BB manufactured by Nikko Chemicals Co., Ltd., and fluorine-based or silicone-based nonionic surfactants such as ZONYL FSN and FSO manufactured by DUPONT, SURFLON S-141 and 145 manufactured by Asahi Glass Company, MEGAFAC F-141 and 144 manufactured by Dainippon Ink & Chemicals, Inc., FTERGENT F-200 and F-251 manufactured by NEOS, UNIDYNE DS-401 and 402 manufactured by Daikin Industries Ltd., FLUORAD FC-170 and 176 manufactured by 3M, and the like, further, cationic surfactants, anionic surfactants and ampholytic surfactants can also be used.

When a physicochemical cell adhesive material is used as of a decomposition substance type as described above, it is usually preferable to use a binder component, separately. The binder component used in this case is not particularly restricted providing it has high bonding energy so that the main skeleton is not decomposed by the action of a photocatalyst. Specifically listed are polysiloxanes having no organic substituent or polysiloxanes having some organic substituents, and these can be obtained by hydrolysis and polycondensation of tetramethoxysilane, tetraethoxysilane and the like.

In this embodiment, a physicochemical cell adhesive material of binder type and a physicochemical cell adhesive material of decomposition substance type as described above may also be used in combination.

There are also physicochemical cell adhesiveness changing materials changing of adhesiveness with cells by controlling of electrostatic interaction. In the case of such materials, a functional group having positive charge contained in the material is decomposed by the action of a photocatalyst in irradiation with energy, and resultantly, the amount of positive charge present in the surface varies, and by this, adhesiveness with cells is changed, to form a cell adhesiveness changing pattern. For example, poly L lysine and the like are listed as such materials.

b. Biological Cell Adhesiveness Changing Material

As biological factors for adhering cells on a base material and culturing cells, there are materials capable of being adhered by a lot of cell species, and materials capable of being adhered only by specific cell species. The former includes, for example, collagen type I, and the latter includes, for example, poly (N-p-vinylbenzyl-[O-β-D-galactopyranosyl-(1→4)-D-glucon amide]) (hereinafter, PVLA) selectively adhering liver parenchymal cells, and the like. In the case of PVLA, it is guessed that by presence of galactose group specifically recognized by a liver parenchymal cell in its structure, selective and specific adhesion between the material and cells is performed.

In the case of mixing such a material with a photocatalyst to give a photocatalyst-containing cell adhesiveness changing layer used, the following use embodiment is envisaged. Collagen type I is solubilized by enzyme treatment to give soluble collagen I which is mixed with previously calcinated and ground TiO₂ particles to obtain a mixture as a photocatalyst-containing cell adhesiveness changing layer material. Next, the photocatalyst-containing cell adhesiveness changing layer material is applied on a base material to form a photocatalyst-containing cell adhesiveness changing layer. When this photocatalyst-containing cell adhesiveness changing layer is irradiated with a small quantity of energy, a part of a cell adhesive peptide structure present at the side chain of collagen can be decomposed, to decrease cell adhesiveness. Further, by increasing energy irradiation quantity, a cell adhesive peptide structure can be gradually deleted, and cell adhesiveness can be further decreased. Furthermore, by conducting excess energy irradiation, the main structure of collagen can be decomposed, to completely delete its cell adhesiveness.

(2) Photocatalyst

Listed as the photocatalyst used in this embodiment are those known as an optical semiconductor, for example, titanium dioxide (TiO₂), zinc oxide (ZnO), tin oxide (SnO₂), strontium titanate (SrTiO₃), tungsten oxide (WO₃), bismuth oxide (Bi₂O₃) and iron oxide (Fe₂O₃), and one or more of them can be selected and mixed for use.

In this embodiment, particularly, titanium dioxide is suitably used since its band gap energy is high, it is chemically stable and not toxic, and easily available. Titanium dioxide involves anatase type and rutile type, and both of them can be used in this embodiment, and preferable is titanium dioxide of anatase type. Titanium dioxide of anatase type has an excitation wavelength of 380 nm or less.

Listed as such titanium dioxide of anatase type are, for example, anatase titania sol of hydrochloric acid peptisation type (STS-02 manufactured by Ishihara Sangyo Kaisha Ltd. (average particle size: 7 nm), ST-K01 manufactured by Ishihara Sangyo Kaisha Ltd.), anatase titania sol of nitric acid peptisation type (TA-15 manufactured by Nissan Chemical Industries, Ltd. (average particle size: 12 nm) and the like.

Smaller particle size of a photocatalyst is preferable since then a photocatalyst reaction occurs effectively, and the average particle size is preferably 50 nm or less, and it is particularly preferable to use a photocatalyst having an average particle size of 20 nm or less.

The content of a photocatalyst in a photocatalyst-containing cell adhesiveness changing layer used in this embodiment can be set in the range from 5 to 60 wt %, preferably from 20 to 40 wt %.

2. Base Material

The base material used in the cell culture base material of the present invention is not particularly restricted providing it is formed of a material capable of forming a photocatalyst-containing cell adhesiveness changing layer on the surface, and its form is not limited providing surface treatment by exposure treatment is possible. Specifically listed are inorganic materials such as metal, glass, silicon and the like and organic materials typified by plastic.

3. Cell Adhesiveness Changing Pattern

In this embodiment, the above-described photocatalyst-containing cell adhesiveness changing layer is formed on the base material, further, irradiated with energy in the form of pattern, to form a cell adhesiveness changing pattern which is a pattern changed adhesiveness with cells.

Such a cell adhesiveness changing pattern is usually formed of a cell adhesiveness excellent region of good cell adhesiveness and a cell adhesiveness inhibited region of poor cell adhesiveness. By adhesion of cells to this cell adhesiveness excellent region, cells can be adhered in the form of highly precise pattern.

Such cell adhesiveness excellent region and cell adhesiveness inhibited region are determined depending on the kind of a cell adhesiveness changing material used.

For example, when the cell adhesiveness changing material is a physicochemical cell adhesiveness changing material which changes adhesiveness of cells by changing surface free energy, the adhesiveness of cells is good under surface free energy within given range, and out of this range, the adhesiveness with cells tends to lower. As the change of adhesiveness of cells by surface free energy as described above, there are known, for example, experiment results as shown in Data, CMC Press, Saisentan of Biomaterial, Yoshito Ikada (ed.), p. 109, lower part.

The adhesiveness of cells can be determined not only depending on the surface free energy of the material but also depending on what kind of cell species is contacted with what kind of material species.

Here, this cell adhesiveness changing pattern may be a pattern of two levels of the cell adhesiveness excellent region and cell adhesiveness inhibited region, and depending on application, the cell adhesiveness changing pattern may be a cell adhesiveness changing pattern having regions differing in surface cell adhesiveness by at least 3 levels or more.

The reason for this is that when, for example, a photocatalyst-containing cell adhesiveness changing layer using a biological cell adhesiveness changing material is used and a condition for excellent cell adhesiveness is not determined yet, and the like, there are sometimes merits such as that a condition suitable for adhesiveness can be found by continuously changing the surface condition of a photocatalyst-containing cell adhesiveness changing layer, and the like.

As described above, in the present invention, the term 3 levels or more includes a condition of continuously changing cell adhesiveness, and which level is used is determined by appropriately selecting depending on situations.

In the case of forming regions of different adhesiveness of multi-level, it can be conducted by changing the irradiation quantity of energy for a photocatalyst-containing cell adhesiveness changing layer. Specifically, a method of using photo masks of half tone different in transmittance, and other methods are exemplified.

Further, in this embodiment, a cell adhesiveness changing pattern can be used utilizing a difference in photocatalyst activity between a part irradiated with energy and a part not irradiated with energy. Namely, for example, in the case of use of a biological cell adhesiveness changing material introduced in a photocatalyst-containing cell adhesiveness changing layer as a decomposition substance, if the surface of the photocatalyst-containing cell adhesiveness changing layer is irradiated with energy in the form of pattern, the biological cell adhesiveness changing material exuded on the surface of the irradiated part is decomposed, and the biological cell adhesiveness changing material on the non-irradiated part remains. Therefore, when this biological cell adhesiveness changing material is a material showing excellent adhesiveness with a specific cell, or a cell showing excellent adhesiveness with a lot of cells, the non-irradiated part becomes a cell adhesiveness excellent region, however, a part irradiated with energy does not carry a biological cell adhesiveness changing material showing excellent adhesiveness with cells, additionally, becomes a region on which a photocatalyst having a sterilization property activated by irradiation with energy is exposed. Therefore, when the part irradiated with energy becomes a cell adhesiveness inhibited region, particularly in the case of culturing for a given period using a cell culture base material of this embodiment, there is a merit that problems such as broadening of a pattern, and the like do not occur.

The energy irradiation method and the like will be illustrated in detail in “II. Method of producing cell culture base material” described later, therefore, explanation thereof is omitted here.

B. Second Embodiment

The second embodiment of the cell culture base material of the present invention is a cell culture base material comprising a base material and a cell adhesiveness changing layer formed on the base material and containing a cell adhesiveness changing material changing of adhesiveness of cells by the action of a photocatalyst in irradiation with energy, wherein on the cell adhesiveness changing layer, a cell adhesiveness changing pattern changed adhesiveness of cells is formed, being characterized in that the cell adhesiveness changing layer has a photocatalyst treating layer containing a photocatalyst and a cell adhesiveness changing material layer formed on the photocatalyst treating layer and containing the cell adhesiveness changing material.

In this embodiment, the cell adhesiveness changing layer has a photocatalyst treating layer formed on a base material, and a cell adhesiveness changing material layer formed on this photocatalyst treating layer as described above, therefore, in irradiating with energy, the cell adhesiveness of a cell adhesiveness changing material in the cell adhesiveness changing material layer changes by the action of a photocatalyst in the photocatalyst treating layer, consequently, a cell adhesiveness changing pattern can be formed differing in cell adhesiveness between parts irradiated with energy and parts not irradiated.

The cell culture base material of this embodiment as described above will be divided into members used and illustrated respectively.

1. Cell Adhesiveness Changing Material Layer

In the cell culture base material of this embodiment, a cell adhesiveness changing material layer is formed on a photocatalyst treating layer formed on a base material. As this cell adhesiveness changing material layer, a layer can be used formed by using a material explained in “(1) Cell adhesiveness changing material” in “1. Photocatalyst-containing cell adhesiveness changing layer” in the first embodiment. Hereinafter, a cell adhesiveness changing material layer using a physicochemical cell adhesiveness changing material and a cell adhesiveness changing material layer using a biological cell adhesiveness changing material will be explained dividedly. (1) Case using physicochemical cell adhesiveness changing material

In this embodiment, the cell adhesiveness changing material layer formed of a physicochemical cell adhesiveness changing material can be a layer using the same material as that described in “(1) Cell adhesiveness changing material” in “1. Photocatalyst-containing cell adhesiveness changing layer” in the first embodiment. The case using such a material is the same as described above excepting the presence or the absence of a photocatalyst, therefore, its explanation will be omitted. In this embodiment, it is basically not necessary that the cell adhesiveness changing material layer contains a photocatalyst, however, a photocatalyst may be contained in small amount from the standpoint of sensitivity.

This embodiment can use a cell adhesiveness changing material layer of type in which a cell adhesiveness changing material layer is formed as a decomposition removal layer decomposed and removed by the action of a photocatalyst on a photocatalyst treating layer, and a region in which the cell adhesiveness changing material layer is decomposed by the action of a photocatalyst in irradiating with energy, namely, a region on which a photocatalyst treating layer is exposed, and a region on which the cell adhesiveness changing material layer remains, are formed, by this composition, a cell adhesiveness changing pattern is obtained.

Specifically, in the case of controlling the adhesiveness of cells by surface free energy, a physicochemical cell adhesiveness changing material having surface free energy suitable for cell adhesiveness is used, this material is applied on the whole surface to form a cell adhesiveness changing material layer, then, irradiated with energy in the form of pattern to form a pattern composed of presence or absence of the cell adhesiveness changing material layer, and this is used as a cell adhesiveness changing pattern.

As the material used in a physicochemical cell adhesiveness changing material layer as a decomposition removal layer and can be used in controlling the adhesiveness of cells by surface free energy as described above, there are listed, for example, regenerated cellulose, nylon 11 and the like.

In the case of controlling the adhesiveness of cells by electrostatic interaction, a physicochemical cell adhesiveness changing material having positive charge is used, and a cell adhesiveness changing pattern can be obtained by the same method as described above.

As the material used in a physicochemical cell adhesiveness changing material layer as a decomposition removal layer and can be used in controlling the adhesiveness of cells by electrostatic interaction as described above, there are exemplified polyamine graft poly(2-hydroxymethyl methacrylate)(HA-x) and the like.

These resins can be dissolved in a solvent, and shaped by a general film forming method such as, for example, spin coat and the like. In the present invention, a film of no defect can be formed by using a functional thin film, namely, self-assembled monolayer, Langmuir-Blodgett's film, alternate adsorption film and the like, therefore, it is more preferable to use such a film formation method.

In the case of formation of a cell adhesiveness changing pattern using a cell adhesiveness changing material layer as a decomposition removal layer as described above, a photocatalyst treating layer described later is exposed in the decomposed and removal region, consequently, this becomes a region on which culturing of cells is significantly inhibited. Therefore, a cell culture base material obtained by such a method has a merit that a highly precise pattern can be maintained even if cells are held for a long period of time.

(2) Case Using Biological Cell Adhesiveness Changing Material

In this embodiment, as the cell adhesiveness changing material layer formed of a biological cell adhesiveness changing material, there are listed, for example, collagen type I described in “(1) Cell adhesiveness changing material” in “1. Photocatalyst-containing cell adhesiveness changing layer” in the first embodiment, and the like.

2. Photocatalyst Treating Layer

Next, the photocatalyst treating layer used in the present invention will be described. The photocatalyst treating layer used in the present invention is not particularly restricted providing it has a composition in which a photocatalyst in the photocatalyst treating layer changes the cell adhesive property of a cell adhesiveness changing material layer formed thereon, and it may be constituted a photocatalyst and a binder, or may be consisted of only a photocatalyst. Its surface may particularly be lyophilic or liquid repellant, and preferable is lyophilic from the standpoint of formation of a cell adhesiveness changing material layer and the like on this photocatalyst treating layer.

Though the action mechanism of a photocatalyst typified by titanium oxide as described later in this photocatalyst treating layer is no necessarily clear, it is believed that a carrier produced by irradiation with light causes a change in chemical structure of an organic substance by direct reaction with a neighboring compound or by active oxygen species generated in the presence of oxygen and water. In the present invention, it is supposed that this carrier acts on a compound in a cell adhesiveness changing material layer formed on a photocatalyst treating layer.

Such a photocatalyst is the same as described in detail in the first embodiment, therefore, explanation thereof is omitted.

The photocatalyst treating layer in this embodiment may be consisted of only a photocatalyst as described above, or may be formed in admixture with a binder.

In the case of a photocatalyst treating layer consisted of only a photocatalyst, an efficiency for the change of the cell adhesive property of a cell adhesiveness changing material layer is improved, and merits from the standpoint of cost such as shortening of treating time and the like are obtained. On the other hand, in the case of a photocatalyst treating layer composed of a photocatalyst and a binder, there is a merit of easiness of formation of the photocatalyst treating layer.

As the method of forming a photocatalyst treating layer consisted of only a photocatalyst, there are listed, for example, vacuum film formation methods such as a sputtering method, CVD method, vacuum deposition method and the like. By forming a photocatalyst treating layer by a vacuum film formation method, a photocatalyst treating layer composed uniform film and consisting of only a photocatalyst can be made, and by this, the property of a cell adhesiveness changing material layer can be changed uniformly, and since the photocatalyst treating layer is consisted of only a photocatalyst, it is made possible to change efficiently the cell adhesiveness of a cell adhesiveness changing layer as compared with the case using a binder.

As other example of forming a photocatalyst treating layer consisted of only a photocatalyst, for example in the case of a photocatalyst consisted of titanium dioxide, there are exemplified a method in which amorphous titania is formed on a base material, then, phase-changed into crystalline titania by calcination, and the like. The amorphous titania herein used can be obtained, for example, by hydrolysis and de-hydration condensation of inorganic salts of titanium such as titanium tetrachloride, titanium sulfate and the like, and hydrolysis and de-hydration condensation of organic titanium compounds such as tetraethoxytitanium, tetraisopropoxytitanium, tetra-n-propoxytitanium, tetrabutoxytitanium, tetramethoxytitanium and the like in the presence of an acid. Then, it can be converted into anatase titania by calcination at 400 to 500° C., and converted into rutile titania by calcination at 600 to 700° C.

When a binder is used, preferable are those having high bonding energy so that the main skeleton thereof is not decomposed by the action of the photocatalyst, and examples of such binders include the organopolysiloxanes and the like.

When an organopolysiloxane is thus used as a binder, the photocatalyst treating layer can be formed by dispersing a photocatalyst and an organopolysiloxane as a binder, if necessary, together with other additives, in a solvent to prepare coating solution, and applying this coating solution on a transparent base material. As the solvent used, alcohol-based organic solvents such as ethanol, isopropanol and the like are preferable. Application can be conducted by known application methods such as spin coat, spray coat, dip coat, roll coat, bead coat and the like. When a component of ultraviolet hardening type is contained as a binder, a photocatalyst treating layer can be formed by hardening treatment by irradiation with ultraviolet ray.

Further, an amorphous silica precursor can be used as a binder. This amorphous silica precursor involves preferably silicon compounds of the general formula: SiX₄ wherein X represents a halogen, methoxy group, ethoxy group, acetyl group or the like, silanols which are hydrolyzates thereof, or polysiloxanes having an average molecular weight of 3000 or less.

Specifically listed are tetraethoxysilane, tetraisopropoxysilane, tetra-n-propoxysilane, tetrabutoxysilane, tetramethoxysilane and the like. In this case, a photocatalyst treating layer can be formed by uniformly dispersing particles of an amorphous silica precursor and a photocatalyst in a non-aqueous solvent, hydrolyzing them with moisture in air to form a silanol on a transparent base material, then, de-hydrating and polycondensing this at normal temperature. When de-hydration and polycondensation of a silanol is conducted at 100° C. or more, the degree of polymerization of the silanol increases, leading to improvement of the strength of the film surface. These binders can be used singly or in admixture of two or more.

The content of a photocatalyst in a photocatalyst treating layer when a binder is used can be set in the range of 5 to 60 wt %, preferably of 20 to 40 wt %. The thickness of a photocatalyst treating layer is preferably in the range of 0.05 to 10 μm.

The photocatalyst treating layer can contain a surfactant, in addition to the photocatalysts and binders. Specifically listed are hydrocarbon-based nonionic surfactants such as NIKKOL BL, BC, BO and BB manufactured by Nikko Chemicals Co., Ltd., and fluorine-based or silicone-based nonionic surfactants such as ZONYL FSN and FSO manufactured by DU PONT, SURFLON S-141 and 145 manufactured by Asahi Glass Company, MEGAFAC F-141 and 144 manufactured by Dainippon Ink & Chemicals, Inc., FTERGENT F-200 and F-251 manufactured by NEOS, UNIDYNE DS-401 and 402 manufactured by Daikin Industries Ltd., FLUORAD FC-170 and 176 manufactured by 3M, and the like, further, cationic surfactants, anionic surfactants and ampholytic surfactants can also be used.

Further, the photocatalyst treating layer can contain oligomers, polymers and the like of polyvinyl alcohol, unsaturated polyesters, acrylic resins, polyethylene, diallyl phthalate, ethylene propylene diene monomer, epoxy resins, phenol resins, polyurethane, melamine resins, polycarbonates, polyvinyl chloride, polyamides, polyimides, styrene butadiene rubber, chloroprene rubber, polypropylene, polybutylene, polystyrene, polyvinyl acetate, polyesters, polybutadiene, polybenzimidazole, polyacrylnitrile, epiochlorohydrin, polysulfide, polyisoprene and the like, in addition to the surfactants.

3. Base Material

The base material used in this embodiment is not particularly restricted providing it can form the photocatalyst treating layer, and the same materials as described in the first embodiment can be used, and explanations thereof are omitted here.

4. Cell Adhesiveness Changing Pattern

In this embodiment, a cell adhesiveness changing pattern is formed which is a pattern of changed adhesiveness with cells on the surface of the cell adhesiveness changing material layer, by the action of a photocatalyst in a photocatalyst treating layer, by irradiating the cell adhesiveness changing material layer with energy in the form of pattern.

Here, the cell adhesiveness changing pattern is the same as described in the first embodiment, and a method of its irradiation with energy and the like will be illustrated in detail in “II. Method of producing cell culture base material” described later, therefore, explanations thereof are omitted here.

C. Third Embodiment

The cell culture base material of this embodiment comprises a base material and a cell adhesiveness changing layer formed on the base material and containing a cell adhesiveness changing material changing of adhesiveness of cells by the action of a photocatalyst in irradiation with energy, wherein on the cell adhesiveness changing layer, a cell adhesiveness changing pattern changed adhesiveness of cells is formed, being characterized in that the cell adhesiveness changing layer is a cell adhesiveness changing material layer containing the cell adhesiveness changing material, and the cell adhesiveness changing pattern is formed by placing the photocatalyst-containing layer containing a photocatalyst and the cell adhesiveness changing material layer so that they face and then irradiating energy from given direction.

In this embodiment, the cell adhesiveness changing layer is a cell adhesiveness changing material layer and the cell adhesiveness changing pattern is formed by placing the photocatalyst-containing layer containing a photocatalyst and the cell adhesiveness changing material layer so that they face and then irradiating energy from given direction, as described above, therefore, when irradiated with energy, the cell adhesiveness of a cell adhesiveness changing material in the cell adhesiveness changing material layer is changed by the action of a photocatalyst in the photocatalyst treating layer, consequently, a cell adhesiveness changing pattern can be formed differing in cell adhesiveness between parts irradiated with energy and parts not irradiated.

The cell culture base material of this embodiment as described above will be divided into members used and illustrated respectively.

1. Cell Adhesiveness Changing Material Layer

In the cell culture base material of this embodiment, a cell adhesiveness changing material layer is formed on a base material. This cell adhesiveness changing material layer is the same as a layer formed by using the material explained in “(1) Cell adhesiveness changing material” in “1. Photocatalyst-containing cell adhesiveness changing layer” in the second embodiment, therefore, explanations thereof will be omitted here. In this embodiment, it is basically not necessary that the cell adhesiveness changing material layer contains a photocatalyst, however, a photocatalyst may be contained in small amount from the standpoint of sensitivity.

In this embodiment, a cell adhesiveness changing material layer may be formed, as a decomposition removal layer to be decomposed and removed by the action of a photocatalyst, on a base material, in the same manner as in the second embodiment as described above. In this case, a cell adhesiveness changing material layer of type is used in which a region in which the cell adhesiveness changing material layer is decomposed by the action of a photocatalyst in irradiating with energy, namely, a region on which a base material is exposed, and a region on which the cell adhesiveness changing material layer remains, are formed, by irradiating with energy using a photocatalyst-containing layer-side base plate, and by this, a cell adhesiveness changing pattern is obtained.

2. Base Material

The base material used in this embodiment is not particularly restricted providing the cell adhesiveness changing material layer can be formed thereof, and the same material as that described in the first embodiment can be used, therefore, explanations thereof will be omitted here.

3. Photocatalyst-containing Layer

Next, the photocatalyst-containing layer used in this embodiment will be illustrated. The photocatalyst-containing layer used in this embodiment is a layer containing a photocatalyst, and usually, formed on a substrate made of glass and the like and used. In this embodiment, such a photocatalyst-containing layer is placed so that it faces the cell adhesiveness changing material layer and irradiated with energy, thus, the cell adhesiveness of the cell adhesiveness changing material layer can be changed by the action of a photocatalyst contained in the photocatalyst-containing layer. This embodiment has a merit that, this photocatalyst-containing layer can be placed at given position in energy irradiation to form a cell adhesiveness changing pattern, therefore, it is not necessary for a photocatalyst to be contained in the cell adhesiveness changing material layer, and the cell adhesiveness changing material layer can be modified so that it does not receive the action of a photocatalyst with the lapse of time.

Such a photocatalyst-containing layer is the same as that described in “2. Photocatalyst-treating layer” in the second embodiment described above, and arrangement of the photocatalyst-containing layer and the cell adhesiveness changing material layer, and irradiation with energy, and the like will be illustrated in detail in the third embodiment of “Method of producing cell culture base material” described later, therefore, explanations thereof will be omitted here.

4. Cell Adhesiveness Changing Pattern

In this embodiment, a cell adhesiveness changing pattern is formed which is a pattern of changed adhesiveness with cells on the surface of the cell adhesiveness changing material layer, by the action of a photocatalyst in a photocatalyst-containing layer, by irradiating the cell adhesiveness changing material layer described above with energy in the form of pattern using the photocatalyst-containing layer.

Here, the cell adhesiveness changing pattern is the same as described in the first embodiment, and a method of its irradiation with energy and the like will be illustrated in detail in “II. Method of producing cell culture base material” described later, therefore, explanations thereof are omitted here.

### II. Method of Producing Cell Culture Base Material

Next, the cell culture base material of the present invention will be illustrated. The method of producing a cell culture base material of the present invention includes three embodiments, and all of these embodiments are characterized in that a pattern forming body base material having a base material and a layer formed on the base material and changing of adhesiveness of cells by the action of a photocatalyst in irradiation with energy is formed, and this pattern forming body base material is irradiated with energy, to allow the photocatalyst to act to form a cell adhesiveness changing pattern having changed cell adhesiveness.

According to the method of producing a cell culture base material of the present invention, the layer changing of adhesiveness of cells by the action of a photocatalyst in irradiation with energy is formed, therefore, by irradiating this layer with energy in the form of pattern required, a cell culture base material carrying a formed cell adhesiveness changing pattern changed cell adhesiveness in the form of highly precise pattern can be easily produced. Consequently, a cell culture base material can be produced by a simple process, without using treating liquid exerting a negative influence on cells, with a highly precise pattern. Since modification of a cell adhesiveness changing material is not required, width of material selection can be enlarged, and a biological cell adhesiveness changing material manifesting specific adhesiveness as described later can be used without problems.

Embodiments of the method of producing the cell culture base material of the present invention will be illustrated below.

A. First Embodiment

First, the first embodiment of the method of producing the cell culture base material of the present invention will be illustrated. The first embodiment of the method of producing the cell culture base material of the present invention comprises

a pattern forming body base material forming process of forming a pattern forming body base material having a base material and a photocatalyst-containing cell adhesiveness changing layer formed on the base material and containing a photocatalyst and a cell adhesiveness changing material changing of adhesiveness of cells by the action of the photocatalyst in irradiation with energy, and

a cell adhesiveness changing pattern forming process of irradiating the photocatalyst-containing cell adhesiveness changing layer with energy to form a cell adhesiveness changing pattern changed adhesiveness of cells of the photocatalyst-containing cell adhesiveness changing layer.

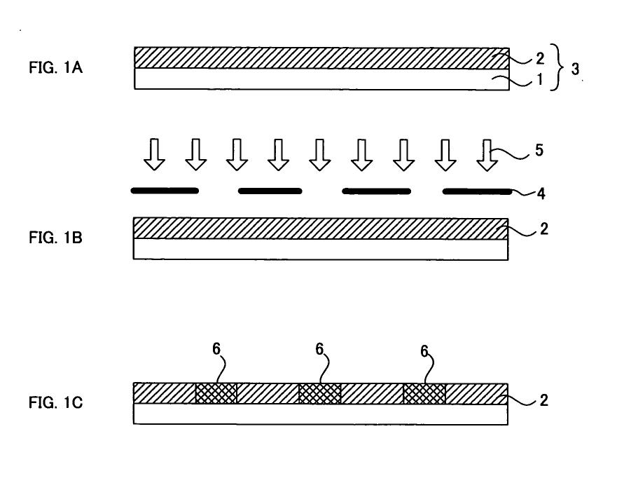

In the method of producing a cell culture base material of this embodiment, first, as shown in FIG. 1 for example, a pattern forming body base material 3 having a base material 1 and a photocatalyst-containing cell adhesiveness changing layer 2 formed on the base material 1 is formed (pattern forming body base material forming process (FIG. 1A)). Next, a cell adhesiveness changing pattern forming process is conducted in which the photocatalyst-containing cell adhesiveness changing layer 2 is irradiated with energy 5 using a photo mask 4 for example (FIG. 1B) to form a cell adhesiveness changing pattern 6 changed adhesiveness of cells of the photocatalyst-containing cell adhesiveness changing layer 2 (FIG. 1C).

In this embodiment, a photocatalyst-containing cell adhesiveness changing layer having a photocatalyst and the cell adhesiveness changing material is formed, therefore, by irradiating with energy in the cell adhesiveness changing pattern forming process, the cell adhesiveness of the cell adhesiveness changing material changes by the action of a photocatalyst in the photocatalyst-containing cell adhesiveness changing layer, consequently, a cell adhesiveness changing pattern can be formed differing in cell adhesiveness between parts irradiated with energy and parts not irradiated. Each process in this embodiment will be illustrated below.

1. Pattern Forming Body Base Material Forming Process

First, the pattern forming body base material forming process in this embodiment will be illustrated. The pattern forming body base material forming process in this embodiment is a process of forming a pattern forming body base material having a base material and a photocatalyst-containing cell adhesiveness changing layer formed on the base material and containing a photocatalyst and a cell adhesiveness changing material changing of adhesiveness of cells by the action of the photocatalyst in irradiation with energy.

This process can be conducted by applying coating solution containing a photocatalyst and a cell adhesiveness changing material on a base material by known application methods such as, for example, spin coat, spray coat, dip coat, roll coat, bead coat and the like, to form a photocatalyst-containing cell adhesiveness changing layer. When a component of ultraviolet hardening type is contained as a binder, a photocatalyst-containing layer can be formed by hardening treatment by irradiation with ultraviolet ray.

Here, regarding the base material, photocatalyst and cell adhesiveness changing material used in the process, the same materials as described in the paragraph of the first embodiment of “I. Cell culture base material” can be used, therefore, explanations thereof will be omitted here.

2. Cell Adhesiveness Changing Pattern Forming Process

Next, the cell adhesiveness changing pattern forming process in this embodiment will be illustrated. The cell adhesiveness changing pattern forming process in this embodiment is a process of irradiating the photocatalyst-containing cell adhesiveness changing layer with energy to form a cell adhesiveness changing pattern changed adhesiveness of cells of the photocatalyst-containing cell adhesiveness changing layer.

In the process, by irradiating with energy in the form of intended pattern, adhesiveness of cells of a photocatalyst-containing cell adhesiveness changing layer can be changed only at a region irradiated with energy, and a cell adhesiveness changing pattern can be formed which is a highly precise pattern composed of a region of excellent cell adhesiveness and a region of poor cell adhesiveness.

Irradiation with energy (exposure) referred to in this embodiment is a concept including irradiation with any energy ray capable of changing cell adhesiveness on the surface of a photocatalyst-containing cell adhesiveness changing layer, and is not limited to irradiation with visible light.

Usually, the wavelength of light used for such energy irradiation is set in the range of 400 nm or less, preferably in the range of 380 nm or less. The reason for this is that a preferable photocatalyst used in a photocatalyst-containing cell adhesiveness changing layer as described above is titanium dioxide, and as energy for activating the photocatalyst action by this titanium dioxide, light of wavelength in the range is preferable.

As a light source used for such energy irradiation, listed are a mercury lamp, metal halide lamp, xenon lamp, excimer lamp, and other various light sources.

It is also possible to use a method of draught-irradiation in the form of pattern using excimer laser, YAG laser and the like, in addition to methods conducted by pattern irradiation via a photo mask, using a light source as described above.

The irradiation quantity of energy in energy irradiation is that necessary for effecting changing of adhesiveness of cells on the surface of a photocatalyst-containing cell adhesiveness changing layer, by the action of a photocatalyst in a photocatalyst-containing cell adhesiveness changing layer.

In this case, irradiation with energy while heating a photocatalyst-containing cell adhesiveness changing layer is preferable since then sensitivity can be increased and efficient change of adhesiveness of cells can be conducted. Specifically, it is preferable to heat at temperatures in the range of 30° C. to 80° C.

Regarding irradiation direction with energy in this embodiment, pattern irradiation with energy or draught irradiation with laser via a photo mask may be conducted from any direction of the base material side and the photocatalyst-containing cell adhesiveness changing layer side, when the base material is transparent. On the other hand, when the base material is not transparent, it is necessary to conduct irradiation with energy from the photocatalyst-containing cell adhesiveness changing layer side.

B. Second Embodiment

Next, the second embodiment of the method of producing the cell culture base material of the present invention will be illustrated. The second embodiment of the method of producing the cell culture base material of the present invention comprises

a pattern forming body base material forming process of forming a pattern forming body base material having a base material, a photocatalyst treating layer containing a photocatalyst formed on the base material and a cell adhesiveness changing material layer formed on the photocatalyst treating layer and containing a cell adhesiveness changing material changing of adhesiveness of cells by the action of the photocatalyst in irradiation with energy, and

a cell adhesiveness changing pattern forming process of irradiating the cell adhesiveness changing material layer with energy to form a cell adhesiveness changing pattern changed adhesiveness of cells of the cell adhesiveness changing material layer.

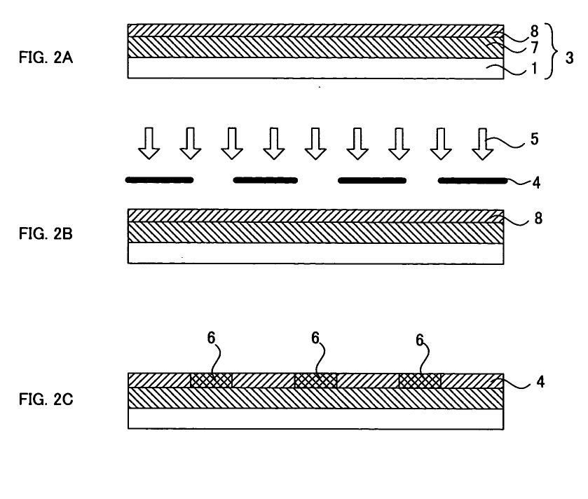

In the method of producing a cell culture base material of this embodiment, first, as shown in FIG. 2 for example, a pattern forming body base material 3 having a base material 1, a photocatalyst treating layer 7 formed on the base material 1 and a cell adhesiveness changing material layer 8 formed on the photocatalyst treating layer 7 is formed (pattern forming body base material forming process (FIG. 2A)). Next, a cell adhesiveness changing pattern forming process is conducted in which the cell adhesiveness changing material layer 8 is irradiated with energy 5 using a photo mask 4 for example (FIG. 2B) to form a cell adhesiveness changing pattern 6 changed adhesiveness of cells of the cell adhesiveness changing material layer 8 (FIG. 2C).

In this embodiment, the photocatalyst treating layer and the cell adhesiveness changing material layer are formed, therefore, by irradiating with energy in the cell adhesiveness changing pattern forming process, cell adhesiveness in the cell adhesiveness changing material layer changes by the action of a photocatalyst contained in the photocatalyst treating layer, consequently, a cell adhesiveness changing pattern can be formed differing in cell adhesiveness between parts irradiated with energy and parts not irradiated. Each process in this embodiment will be illustrated below.

1. Pattern Forming Body Base Material Forming Process

First, the pattern forming body base material forming process in this embodiment will be illustrated. The pattern forming body base material forming process in this embodiment is a process of forming a pattern forming body base material having a photocatalyst treating layer containing a photocatalyst formed on the base material and a cell adhesiveness changing material layer formed on the photocatalyst treating layer and containing a cell adhesiveness changing material changing of adhesiveness of cells by the action of the photocatalyst in irradiation with energy.

The photocatalyst treating layer formed in this process may be consisted of only a photocatalyst or may be formed in admixture with a binder.

As the method of forming a photocatalyst treating layer consisted of only a photocatalyst, there are listed, for example, vacuum film formation methods such as a sputtering method, CVD method, vacuum deposition method and the like, and when the photocatalyst is titanium dioxide, a method in which amorphous titania is formed on a base material, then, it is converted into crystalline titania by calcination, and other methods are exemplified. By forming a photocatalyst treating layer by a vacuum film formation method, a photocatalyst treating layer which is a uniform film and consisting of only a photocatalyst can be obtained, and by this, adhesiveness of cells on a cell adhesiveness changing material layer can be changed uniformly, and since it is consisted of a photocatalyst, adhesiveness of cells on a cell adhesiveness changing material layer can be changed efficiently as compared with the case of using a binder.

When a photocatalyst treating layer is obtained by mixing the photocatalyst and a binder, the photocatalyst treating layer can be formed by dispersing a photocatalyst and a binder, if necessary, together with other additives, in a solvent to prepare coating solution, and applying this coating solution on a transparent base material. As the solvent used, alcohol-based organic solvents such as ethanol, isopropanol and the like are preferable. Application can be conducted by known application methods such as spin coat, spray coat, dip coat, roll coat, bead coat and the like. When a component of ultraviolet hardening type is contained as a binder, a photocatalyst treating layer can be formed by hardening treatment by irradiation with ultraviolet ray.

Subsequently, the coating solution containing the cell adhesiveness changing material can be applied on the photocatalyst treating layer by known application methods such as spin coat, spray coat, dip coat, roll coat, bead coat and the like to form a cell adhesiveness changing material layer. When a component of ultraviolet hardening type is contained as a binder, a photocatalyst treating layer can be formed by hardening treatment by irradiation with ultraviolet ray.

Here, regarding the base material, photocatalyst treating layer and cell adhesiveness changing material used in the process, the same materials as described in the paragraph of the second embodiment of “I. Cell culture base material” can be used, therefore, explanations thereof will be omitted here.

2. Cell Adhesiveness Changing Pattern Forming Process

Next, the cell adhesiveness changing pattern forming process in this embodiment will be illustrated. The cell adhesiveness changing pattern forming process in this embodiment is a process of irradiating the cell adhesiveness changing material layer with energy to form a cell adhesiveness changing pattern changed adhesiveness of cells of the cell adhesiveness changing material layer.

In this process, by irradiating with energy in the form of intended pattern, adhesiveness of cells of a cell adhesiveness changing material layer can be changed only at a region irradiated with energy, and a cell adhesiveness changing pattern can be formed which is a highly precise pattern composed of a region of excellent cell adhesiveness and a region of poor cell adhesiveness.

The energy irradiation method and energy irradiated in this process are the same as described in the first embodiment, therefore, explanations thereof will be omitted here.

C. Third Embodiment

Next, the third embodiment of the method of producing the cell culture base material of the present invention will be illustrated. The third embodiment of the method of producing the cell culture base material of the present invention comprises

a pattern forming body base material forming process of forming a pattern forming body base material having a base material, and a cell adhesiveness changing material layer formed on the base material and containing a cell adhesiveness changing material changing of adhesiveness of cells by the action of a photocatalyst in irradiation with energy, and

a cell adhesiveness changing pattern forming process of placing the pattern forming body base material and a photocatalyst-containing layer-side base plate having a substrate and a photocatalyst-containing layer containing a photocatalyst so that the cell adhesiveness changing material layer and the photocatalyst-containing layer face, then, irradiating with energy from given direction to form a cell adhesiveness changing pattern changed adhesiveness of cells of the cell adhesiveness changing material layer.

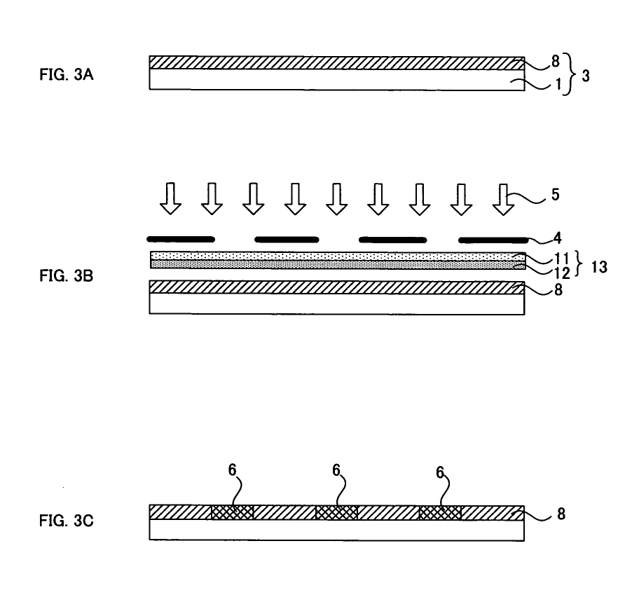

In the method of producing a cell culture base material of this embodiment, first, as shown in FIG. 3 for example, a pattern forming body base material 3 having a base material 1 and a cell adhesiveness changing material layer 8 formed on the base material 1 is formed (pattern forming body base material forming process (FIG. 3A)). Next, a photocatalyst-containing layer-side base plate 13 having a base material 11 and a photocatalyst-containing layer 12 formed on the base material 11 is prepared. A cell adhesiveness changing pattern forming process is conducted in which they were arranged so that the photocatalyst-containing layer 12 on this photocatalyst-containing layer-side base plate 13 and the cell adhesiveness changing material layer 8 face, irradiated with energy 5 using a photo mask 4 for example (FIG. 3B) to form a cell adhesiveness changing pattern 6 changed adhesiveness of cells of the cell adhesiveness changing material layer 8 (FIG. 3C).

In this embodiment, the cell adhesiveness changing material layer is formed, therefore, by irradiating with energy using a photocatalyst-containing layer-side base plate in the cell adhesiveness changing pattern forming process, cell adhesiveness in the cell adhesiveness changing material layer changes by the action of a photocatalyst contained in the photocatalyst-containing layer, consequently, a cell adhesiveness changing pattern can be formed differing in cell adhesiveness between parts irradiated with energy and parts not irradiated. Each process in this embodiment will be illustrated below.

1. Pattern Forming Body Base Material Forming Process

First, the pattern forming body base material forming process in the present invention will be illustrated. The pattern forming body base material forming process in the present invention is a process of forming a pattern forming body base material having a base material and a cell adhesiveness changing material layer formed on the base material and containing a cell adhesiveness changing material changing of adhesiveness of cells by the action of the photocatalyst in irradiation with energy.

This process can be conducted by applying coating solution containing a cell adhesiveness changing material on a base material by known application methods such as, for example, spin coat, spray coat, dip coat, roll coat, bead coat and the like, to form a cell adhesiveness changing material layer. When a component of ultraviolet hardening type is contained as a binder, a photocatalyst-containing layer can be formed by hardening treatment by irradiation with ultraviolet ray.

Here, regarding the base material and cell adhesiveness changing material used in the process, the same materials as described in the paragraph of the first embodiment of “I. Cell culture base material” can be used, therefore, explanations thereof will be omitted here.

2. Cell Adhesiveness Changing Pattern Forming Process

Next, the cell adhesiveness changing pattern forming process in this embodiment will be illustrated. The cell adhesiveness changing pattern forming process in this embodiment is a process of placing the pattern forming body base material and a photocatalyst-containing layer-side base plate having a substrate and a photocatalyst-containing layer containing a photocatalyst so that the cell adhesiveness changing material layer and the photocatalyst-containing layer face, then, irradiating with energy from given direction to form a cell adhesiveness changing pattern changed adhesiveness of cells of the cell adhesiveness changing material layer.

In the process, by placing the photocatalyst-containing layer of the photocatalyst-containing layer-side base plate and the cell adhesiveness changing material layer so that they face and irradiating energy in the form of intended pattern, adhesiveness of cells of a cell adhesiveness changing material layer can be changed only at a region irradiated with energy, and a cell adhesiveness changing pattern can be formed which is a highly precise pattern composed of a region of excellent cell adhesiveness and a region of poor cell adhesiveness.

The photocatalyst-containing layer-side base plate and energy irradiation used in this process will be illustrated below.

(1) Photocatalyst-containing Layer-side Base Plate

The photocatalyst-containing layer-side base plate used in this embodiment will be illustrated.

The photocatalyst-containing layer-side base plate used in this embodiment has at least a photocatalyst-containing layer and a substrate, and usually has a photocatalyst-containing layer in the form of thin film formed by a given method on a substrate. As this photocatalyst-containing layer-side base plate, that carrying a photocatalyst-containing layer-side shielding part or primer layer formed in the form of pattern can also be used.

In this embodiment, in irradiating with energy, the cell adhesiveness changing material layer and the photocatalyst-containing layer of the photocatalyst-containing layer-side base plate are allowed to face at given distance, and by the action of the photocatalyst-containing layer of the photocatalyst-containing layer-side base plate, the cell adhesiveness of the cell adhesiveness changing material layer is changed, and after irradiation with energy, the photocatalyst-containing layer-side base plate is removed to form a cell adhesiveness changing pattern. Hereinafter, compositions of this photocatalyst-containing layer-side base plate will be illustrated.

a. Photocatalyst-containing Layer

The photocatalyst-containing layer used in this embodiment contains at least a photocatalyst, and may or may not contain a binder, and is the same as the photocatalyst treating layer in the second embodiment described above, therefore, explanations thereof will be omitted here.

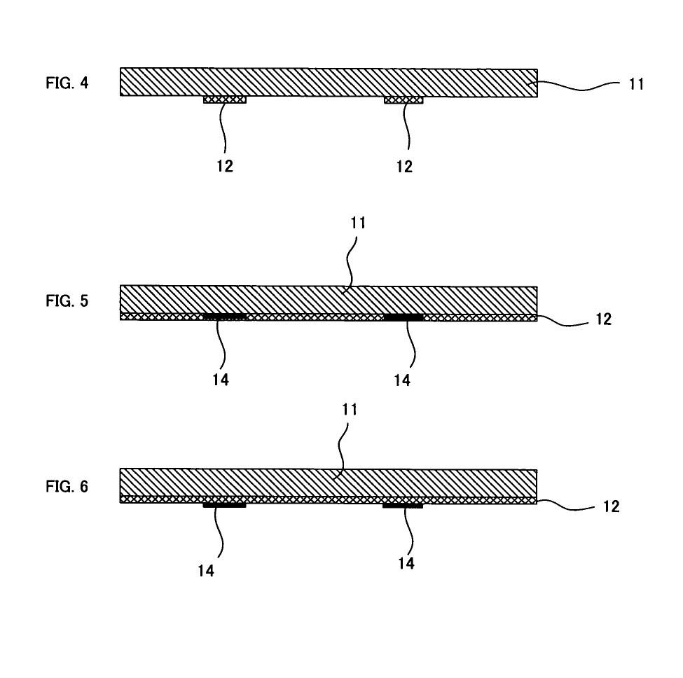

Here, the photocatalyst-containing layer used in this embodiment may be, as shown in FIG. 3 for example, formed on the whole surface of the substrate 11, and for example, the photocatalyst-containing layer 12 may be formed in the form of pattern on the substrate 11, as shown in FIG. 4.

By thus forming a photocatalyst-containing layer in the form of pattern, it is not necessary to use a photo mask and the like in pattern irradiation in irradiating with energy, and by irradiation on the whole surface, a cell adhesiveness changing pattern can be formed on a cell adhesiveness changing material layer.

This method of patterning of a photocatalyst-containing layer is not particularly restricted, and can be conducted by, for example, a photolithography method and the like.

When a photocatalyst-containing layer and a cell adhesiveness changing material layer are for example closely adhered and irradiated with energy, the property of only a part on which a photocatalyst-containing layer is actually formed changes, therefore, when a part at which the photocatalyst-containing layer and cell adhesiveness changing material layer faces is irradiated with energy, energy irradiation may be conducted from any direction, further, energy used for irradiation is not limited to parallel energy such as parallel light and the like.

b. Substrate

In this embodiment, the photocatalyst-containing layer-side base plate 13 has at least the substrate 11 and the photocatalyst-containing layer 12 formed on this substrate 11. In this case, the material constituting the substrate used is appropriately selected depending on irradiation direction with energy described later, and necessity of transparency of the resulting cell culture base material, and the like.

The substrate used in this embodiment may be that having flexibility, for example, a resin film and the like, or that having no flexibility, for example, a glass base material and the like. Further, as the substrate of another form, optical guide optical guide such as an optical fiber and the like can also be used. These are appropriately selected depending on the energy irradiation method.

For improving close adherence between the surface of a substrate and a photocatalyst-containing layer, an anchor layer may be formed on a substrate. As such an anchor layer, there are listed, for example, silane-based and titanium-based coupling agents and the like.

c. Photocatalyst-containing Layer-side Shielding Part

As the photocatalyst-containing layer-side base plate used in this embodiment, that carrying a photocatalyst-containing layer-side shielding part formed in the form of pattern thereon may be used. By using a photocatalyst-containing layer-side base plate having a photocatalyst-containing layer-side shielding part as described above, it is not necessary to use a photo mask and conduct draught irradiation with laser light, in irradiating with energy. Accordingly, positioning of a photocatalyst-containing layer-side base plate and a photo mask is unnecessary, therefore, a simple process can be established, and an expensive apparatus necessary for draught irradiation is not required, leading to an advantage from the standpoint of cost.

The photocatalyst-containing layer-side base plate having such a photocatalyst-containing layer-side shielding part includes the following two embodiments depending on the position of forming the photocatalyst-containing layer-side shielding part.

One is an embodiment in which, as shown in FIG. 5 for example, the photocatalyst-containing layer-side shielding part 14 is formed on the substrate 11, and the photocatalyst-containing layer 12 is formed on this photocatalyst-containing layer-side shielding part 14, to give a photocatalyst-containing layer-side base plate. Another is an embodiment in which, as shown in FIG. 6 for example, the photocatalyst-containing layer 12 is formed on the substrate 11, and the photocatalyst-containing layer-side shielding part 14 is formed on this, to give a photocatalyst-containing layer-side base plate.

In any embodiment, a photocatalyst-containing layer-side shielding part is placed at a site nearer to a placing part of the photocatalyst-containing layer and cell adhesiveness changing material layer as compared with the case using a photo mask, an influence by scattering of energy in a substrate and the like can be decreased, consequently, pattern irradiation with energy can be conducted extremely correctly.

The embodiment of forming a photocatalyst-containing layer-side shielding part on a photocatalyst-containing layer has a merit that, in placing a photocatalyst-containing layer and a cell adhesiveness changing material layer at a given position, if the film thickness of this photocatalyst-containing layer-side shielding part is allowed to coincide with the width of this gap, the photocatalyst-containing layer-side shielding part can also be used as a spacer for making the gap constant. When the height as a spacer is deficient, a spacer may be separately provided at the shielding part.

Namely, in placing the photocatalyst-containing layer and cell adhesiveness changing material layer at a given gap so that they face, if the photocatalyst-containing layer-side shielding part and cell adhesiveness changing material layer are placed in closely adhered condition, the give gap can be set correctly, and by irradiating energy from the photocatalyst-containing layer-side base plate under this condition, a cell adhesiveness changing pattern can be formed with good precision on the cell adhesiveness changing material layer.

The method of forming such a photocatalyst-containing layer-side shielding part is not particularly restricted, and appropriately selected depending on the property of a surface on which a photocatalyst-containing layer-side shielding part is formed, a shielding property for energy required, and the like.

For example, a thin film of a metal such as chromium and the like having a thickness of about 1000 to 2000 Å may be formed by a sputtering method, vacuum deposition method and the like, and patterned to give a photocatalyst-containing layer-side shielding part. As this patterning method, usual patterning methods such as sputtering and the like can be used.

Further, it may be a method in which a layer containing shielding particles such as carbon fine particles, metal oxides, inorganic pigments, organic pigments and the like in a resin binder is formed into a pattern. Used as the resin binder are mixtures of one or more resins such as polyimide resins, acrylic resins, epoxy resins, polyacrylamide, polyvinyl alcohol, gelatin, casein, cellulose and the like; and photosensitive resins, further, O/W emulsion type resin compositions, for example, those obtained by emulsifying a reactive silicone, and the like. The thickness of such a resin shielding part can be set in the range from 0.5 to 10 μm. As the method of patterning such a resin shielding part, generally used methods such as a photolithography method, printing method and the like can be used.

In the explanations, two cases have been described in which the formation position of a photocatalyst-containing layer-side shielding part is between a substrate and a photocatalyst-containing layer, and on the surface of a photocatalyst-containing layer, however, additionally, an embodiment can also be adopted in which a photocatalyst-containing layer-side shielding part is formed on the surface of the side of a substrate on which a photocatalyst-containing layer is not formed. In this embodiment, there is envisaged, for example, a case in which a photo mask is closely adhered to this surface to a detachable extent or the like, and it can be suitably used when a cell adhesiveness changing pattern is varied every small lot.

d. Primer Layer

Next, a primer layer used for the photocatalyst-containing layer-side base plate of this embodiment will be illustrated. In this embodiment, when a photocatalyst-containing layer-side shielding part is formed in the form of pattern on a substrate and a photocatalyst-containing layer is formed thereon to give a photocatalyst-containing layer-side base plate as described above, a primer layer may be formed between the photocatalyst-containing layer-side shielding part and photocatalyst-containing layer.

Thought the action and function of this primer layer are not necessarily apparent, it is considered that by forming a primer layer between a photocatalyst-containing layer-side shielding part and a photocatalyst-containing layer, the primer layer shows a function of preventing diffusion of impurities from the photocatalyst-containing layer-side shielding part and from apertures present between the photocatalyst-containing layer-side shielding parts which are factors inhibiting change of cell adhesiveness of a cell adhesiveness changing material layer by the action of a photocatalyst, particularly, impurities such as residues generated in patterning the photocatalyst-containing layer-side shielding part, and metals, metal ions and the like. Therefore, by forming a primer layer, treatment of cell adhesiveness changing progresses with high sensitivity, and resultantly, a pattern of high resolution can be obtained.

The primer layer in this embodiment is formed to prevent impurities present not only on a photocatalyst-containing layer-side shielding part but also in apertures formed in a photocatalyst-containing layer-side shielding part from influencing on the action of a photocatalyst, therefore, it is preferable that a primer layer is formed over the whole surface of a photocatalyst-containing layer-side shielding part including apertures.

The primer layer in this embodiment is not particularly restricted providing a structure is established in which a primer layer is formed so that a photocatalyst-containing layer-side shielding part of a photocatalyst-containing layer-side base plate and a photocatalyst-containing layer do not come into contact.

The material constituting this primer layer is not particularly restricted, and preferable are inorganic materials not easily decomposed by the action of a photocatalyst. Specifically listed is amorphous silica. When such amorphous silica is used, the precursor of this amorphous silica involves silicon compounds of the general formula: SiX₄ wherein X represents a halogen, methoxy group, ethoxy group, acetyl group or the like, preferably, silanols which are hydrolyzates thereof, or polysiloxanes having an average molecular weight of 3000 or less.

The thickness of the primer layer is preferably in the range from 0.001 μm to 1 μm, and particularly preferably in the range from 0.001 μm to 0.1 μm.

(2) Energy Irradiation

Next, irradiation with energy in this process will be illustrated. In this embodiment, the cell adhesiveness changing material layer and the photocatalyst-containing layer on the photocatalyst-containing layer-side base plate are placed so that they face and irradiated with energy from given direction, thus, a pattern changed cell adhesiveness of the cell adhesiveness changing material layer can be formed.

The placing means a condition of placement in which the action of a photocatalyst is substantially influenced on the surface of a cell adhesiveness changing material layer, and involves placement of the photocatalyst-containing layer and cell adhesiveness changing material layer at a given gap in addition to a condition of actual physical contact thereof. This gap is preferably 200 μm or less.

The gap in this embodiment is particularly in the range from 0.2 μm to 10 μm, preferably in the range from 1 μm to 5 μm in view that pattern precision is extremely excellent, the sensitivity of a photocatalyst is high, and consequently, efficiency of change of cell adhesiveness of a cell adhesiveness changing material layer is excellent. Such a gap range is particularly effective for a cell adhesiveness changing material layer of small area for which gap can be controlled with high precision.

On the other hand, when treatment is conducted on a cell adhesiveness changing material layer of large area of, for example, 300 mm×300 mm or more, it is extremely difficult to form small gap as described above between a photocatalyst-containing layer-side base plate and a cell adhesiveness changing material layer. Therefore, when a cell adhesiveness changing material layer has relatively large area, the gap is preferably in the range from 10 to 100 μm, particularly in the range from 10 to 20 μm. The reason for this is that by controlling the gap in this range, problems regarding decrease in pattern precision such as diffused pattern and the like and problems of deterioration of the sensitivity of a photocatalyst to worsen efficiency of change of cell adhesiveness, and the like are not caused, further, an effect that irregularity does not occur in change of cell adhesiveness on a cell adhesiveness changing material layer is obtained.

In irradiating a cell adhesiveness changing material layer of relatively large area with energy as described above, the gap in an apparatus for positioning a photocatalyst-containing layer-side base plate and a cell adhesiveness changing material layer in an energy irradiation apparatus is set in the range preferably from 10 μm to 200 μm, particularly from 10 μm to 20 μm. The reason for this is that by setting the gap in such a range, a photocatalyst-containing layer-side base plate and a cell adhesiveness changing material layer can be placed without mutual contact, without causing significant decrease in pattern precision or significant deterioration of the sensitivity of a photocatalyst.

By thus placing a photocatalyst-containing layer and a cell adhesiveness changing material layer surface separately at given distance, active oxygen species generated from oxygen and water and by photocatalysis action can be detached easily. Namely, when the distance between a photocatalyst-containing layer and a cell adhesiveness changing material layer is narrower than the above range, the active oxygen species cannot be detached easily, resultantly, there is a possibility of decreasing a cell adhesiveness changing speed, undesirably. When they are placed at a distance larger than the above range, the generated active oxygen species does not easily reach the cell adhesiveness changing material layer, and also in this case, there is a possibility of decreasing a cell adhesiveness changing speed, undesirably.

As the method of placing a photocatalyst-containing layer and a cell adhesiveness changing material layer with such extremely narrow distance regulated uniformly, there is, for example, a method of using a spacer. By using a spacer as described above, a uniform gap can be formed, and at the same time, regarding a portion to which this spacer contacts, the action of a photocatalyst does not reach the surface of a cell adhesiveness changing material layer, therefore, by allowing this spacer to have the same pattern as the cell adhesiveness changing pattern, a given cell adhesiveness changing pattern can be formed on the cell adhesiveness changing material layer.

In this embodiment, it may be advantageous that such a placing condition is maintained at least only during irradiation with energy.

Here, the kind of energy used for irradiation, irradiation method and the like are the same as described in the column of cell adhesiveness changing pattern forming process in the first embodiment described above, therefore, explanations thereof will be omitted here.

The present invention is not limited to the embodiments. The embodiments are only examples, and any embodiments having substantially the same constitution as the technological idea described in Claims of the present invention and performing the same action and effect are involved in the technological range of the present invention.

### EXAMPLES

The present invention will be illustrated further specifically referring to the following examples.

### Example 1

3 g of isopropyl alcohol, 0.4 g of organosilane TSL 8113 (manufactured by Toshiba Silicone), 0.04 g of fluoroalkylsilane MF-160 E (manufactured by Totem Products) and 1.5 g of a photocatalyst inorganic coating agent ST-K01 (manufactured by Ishihara Sangyo Kaisha, Ltd.) were mixed and heated at 100° C. for 20 minutes while stirring.

This solution was applied on a soda glass base material on which alkali treatment had been performed by a spin coating method, this base material was dried for 10 minutes at a temperature of 150° C. to progress hydrolysis and polycondensation reaction thereof, to obtain a pattern forming body base material on which a photocatalyst-containing cell adhesiveness change layer having a film thickness of 0.2 μm had been formed obtained by strongly fixing a photocatalyst in the organopolysiloxane.

Next, on the photocatalyst-containing cell adhesiveness changing layer on this pattern forming body base material, a pattern surface of a negative photo mask having net points having a diameter of 50 μm placed at a distance of 50 μm was superposed, and exposed to ultraviolet ray at an illuminance of 300 mW/cm² for 900 seconds by a mercury lamp (wavelength: 365 nm) from the photo mask side, to obtain a cell culture base material having a cell adhesiveness changing pattern.

Next, rib cartilage of Lewis 4 weeks male rat was excised and chopped to about 5 mm. Then, the sample was immersed in 0.1% ethylenediamine tetraacetic acid (manufactured by Nacalai Tesque, Inc.)(hereinafter, EDTA)/PBS(−) (manufactured by Roman Industries Co. Ltd.) at 37° C. for 20 minutes, further, immersed in 0.1% EDTA/PBS containing 0.25% trypsin (manufactured by GIBCO at 37° C. for 1 hour. This trypsin-treated cartilage fragment was washed with PBS three times, and immersed in 0.1% collagenase (manufactured by Wako Pure Chemical Industries Ltd.)/PBS (+) at 37° C. for 3 hours, then, washed with PBS, to obtain cartilage cell liquid.

Subsequently, the cell culture base material used was sterilized by washing with a 70% ethanol aqueous solution as pre-treatment of culturing, then, washed three times with PBS.

Next, the cartilage cell liquid was disseminated on the surface of the photocatalyst-containing cell adhesiveness change layer of the cell culture base material, and allowed to stand still in Dulbecco's conditioned Eagle medium (GIBCO) (hereinafter, DMEM) containing 10% FBS and culture for 1 week.

The cell culture base material after cell culture was observed by an optical microscope, to confirm that the cartilage cells were adhered and cultured only on a part treated with ultraviolet ray of the cell culture base material, and the cells were not adhered and cultured on a part not treated.

### Example 2

100 ml of Cellmatrix Type I-C (Nitta Gelatin Inc.) to which 10 g of 5 times concentration DMEM and 10 g of buffer for reconstitution had been previously added, and 1.0 g of a photocatalyst inorganic coating agent ST-K01 (manufactured by Ishihara Sangyo Kaisha Ltd.) were stirred to mix for 20 minutes while cooling to 4° C.

Next, this solution was applied on a soda glass base material on which alkali treatment had been previously performed by a spin coating method, and the base material was subjected to incubation treatment at 37° C. for 10 minutes, to progress gelling. Further, the base material was subjected to freeze dry treatment, to give a pattern forming body base material having a photocatalyst-containing cell adhesiveness changing layer having a film thickness of 0.2 μm obtained by dispersing the photocatalyst in collagen.

On this pattern forming body base material, patterning of the photocatalyst-containing cell adhesiveness changing layer and dissemination and culturing of cells were conducted in the same manners as in Example 1.

As a result, the cell culture base material after cell culture was observed by an optical microscope, to confirm that the cartilage cells were adhered and cultured only on a part treated with ultraviolet ray of the cell culture base material, and the cells were not adhered and cultured on a part not treated.

### Example 3

A pattern forming body base material having a photocatalyst-containing cell adhesiveness changing layer was produced in the same manner as in Example 1.

On the photocatalyst-containing cell adhesiveness changing layer on this pattern forming body base material, a pattern surface of a negative photo mask having a single spot having a diameter of 1000 μm was superposed, and exposed to ultraviolet ray at an illuminance of 300 mW/cm² for 50 seconds by a mercury lamp (wavelength: 365 nm) from the photo mask side. Then, the position of the photo mask was shifted, sites different from the site previously exposed were irradiated with ultraviolet ray at an illuminance of 300 mW/cm² for 100 seconds and 600 seconds respectively, to obtain three spots of different ultraviolet irradiation quantities on the base material, giving a cell culture base material.

On this cell culture base material, dissemination and culturing of cells were conducted in the same manners as in Example 1, and the number of cells on the cell culture base material was confirmed by a counting board through observation by an optical microscope, to find that cells were not adhered to the spot irradiated for 50 seconds, and 3.3×10³ cells and 4.8×10⁴ cells were adhered to and cultured on the spot of 100 seconds and a spot of 600 seconds respectively in the form corresponding to the exposure pattern.

### Example 4

First, 5 g of trimethoxymethylsilane TSL 8113 (GE Toshiba Silicone) and 2.5 g of 0.5 N hydrochloric acid were mixed and stirred for 8 hours. This was diluted 10 times with isopropyl alcohol, to obtain a composition for anchor layer.

The composition for anchor layer was applied by a spin coating method on a soda glass base material on which alkali treatment had been previously conducted, and the base material was dried at a temperature of 150° C. for 10 minutes, to obtain a soda glass base material having the anchor layer.

Next, 30 g of isopropyl alcohol, 3 g of trimethoxymethylsilane TSL 8113 (GE Toshiba Silicone) and 20 g of a photocatalyst inorganic coating agent ST-K03 (manufactured by Ishihara Sangyo Kaisha, Ltd.) were mixed and stirred at 100° C. for 20 minutes. This was diluted 3 times with isopropyl alcohol to obtain a composition for photocatalyst treating layer.

This composition for photocatalyst treating layer was applied by a spin coating method on the anchor layer surface of the soda glass base material, and the base material was dried at a temperature of 150° C. for 10 minutes to obtain a pattern forming body base material having a photocatalyst treating layer.

Next, 3 g of isopropyl alcohol, 0.4 g of organosilane TSL 8113 (manufactured by Toshiba Silicone) and 0.04 g of fluoroalkylsilane MF-160 E (manufactured by Tokem Products) were mixed and heated at 100° C. for 20 minutes while stirring.

This solution was applied by a spin coating method on the surface of the photocatalyst treating layer of the base material, and this base material was dried for 10 minutes at a temperature of 150° C. to progress hydrolysis and polycondensation reaction thereof, to obtain a pattern forming body base material having the cell adhesiveness changing material layer and photocatalyst treating layer.

On this pattern forming body base material, patterning of the base material and dissemination and culturing of cells were conducted in the same manners as in Example 1.

As a result, the cell culture base material after cell culture was observed by an optical microscope, to confirm that the cartilage cells were adhered and cultured only on a part treated with ultraviolet ray of the cell culture base material, and the cells were not adhered and cultured on a part not treated.

### Example 5

3 g of isopropyl alcohol, 0.4 g of organosilane TSL 8113 (manufactured by Toshiba Silicone) and 0.04 g of fluoroalkylsilane MF-160 E (manufactured by Tokem Products) were mixed and heated at 100° C. for 20 minutes while stirring.

Next, this solution was applied by a spin coating method on a soda glass base material on which alkali treatment had been previously conducted, and the base material was dried at a temperature of 150° C. for 10 minutes to progress hydrolysis and polycondensation reaction, to obtain a pattern forming body base material on which a cell adhesiveness changing material layer having a film thickness of 0.2 μm had been formed.

Next, 5 g of trimethoxymethylsilane TSL 8113 (GE Toshiba Silicone) and 2.5 g of 0.5 N hydrochloric acid were mixed and stirred for 8 hours. This was diluted with isopropyl alcohol 10 times, to give a composition for anchor layer.

The composition for anchor layer was applied by a spin coating method on the pattern surface of a negative photo mask on which net points having a diameter of 50 μm had been placed at a distance of 50 μm, and the base material was dried at a temperature of 150° C. for 10 minutes, to obtain a photo mask having an anchor layer.

Next, 30 g of isopropyl alcohol, 3 g of trimethoxymethylsilane TSL 8113 (GE Toshiba Silicone) and 20 g of a photocatalyst inorganic coating agent ST-K03 (manufactured by Ishihara Sangyo Kaisha, Ltd.) were mixed and stirred at 100° C. for 20 minutes. This was diluted 3 times with isopropyl alcohol to obtain a composition for photocatalyst-containing layer.

The composition for photocatalyst-containing layer was applied by a spin coater on the photo mask on which the anchor layer had been formed, and subjected to drying treatment at 150° C. for 10 minutes, to form a photo mask having a transparent photocatalyst-containing layer.

The photocatalyst-containing layer surface of the photo mask and the cell adhesiveness changing material layer surface of the base material was place at a distance of 10 μm, and exposed to ultraviolet ray at an illuminance of 300 mW/cm² for 900 seconds by a mercury lamp (wavelength: 365 nm) from the photo mask side, to obtain a cell culture base material having a cell adhesiveness changing pattern.

Subsequently, on this cell culture base material, dissemination and culturing of cells were conducted in the same manners as in Example 1.

As a result, the cell culture base material after cell culture was observed by an optical microscope, to confirm that the cartilage cells were adhered and cultured only on a part treated with ultraviolet ray of the cell culture base material, and the cells were not adhered and cultured on a part not treated.

What is claimed is:

1. A cell culture base material comprising a base material and a cell adhesiveness changing layer formed on the base material and containing a cell adhesiveness changing material changing of adhesiveness of cells by the action of a photocatalyst in irradiation with energy, wherein on the cell adhesiveness changing layer, a cell adhesiveness changing pattern changed adhesiveness of cells is formed.

2. The cell culture base material according to claim 1, wherein the cell adhesiveness changing layer is a photocatalyst-containing cell adhesiveness changing layer containing a photocatalyst and the cell adhesiveness changing material.

3. The cell culture base material according to claim 1, wherein the cell adhesiveness changing layer has a photocatalyst treating layer containing a photocatalyst and a cell adhesiveness changing material layer formed on the photocatalyst treating layer and containing the cell adhesiveness changing material.

4. The cell culture base material according to claim 1, wherein the cell adhesiveness changing layer is a cell adhesiveness changing material layer containing the cell adhesiveness changing material, and the cell adhesiveness changing pattern is formed by placing the photocatalyst-containing layer containing a photocatalyst and the cell adhesiveness changing material layer so that they face and then irradiating energy from given direction.

5. The cell culture base material according to claim 1, wherein the cell adhesiveness changing pattern is a cell adhesiveness changing pattern having regions differing in surface cell adhesiveness by at least 3 levels or more.

6. A method of producing a cell culture base material comprising:
a pattern forming body base material forming process of forming a pattern forming body base material having a base material and a photocatalyst-containing cell adhesiveness changing layer formed on the base material and containing a photocatalyst and a cell adhesiveness changing material changing of adhesiveness of cells by the action of the photocatalyst in irradiation with energy; and
a cell adhesiveness changing pattern forming process of irradiating the photocatalyst-containing cell adhesiveness changing layer with energy to form a cell adhesiveness changing pattern changed adhesiveness of cells of the photocatalyst-containing cell adhesiveness changing layer.

7. A method of producing a cell culture base material comprising:
a pattern forming body base material forming process of forming a pattern forming body base material having a base material, a photocatalyst treating layer containing a photocatalyst formed on the base material and a cell adhesiveness changing material layer formed on the photocatalyst treating layer and containing a cell adhesiveness changing material changing of adhesiveness of cells by the action of the photocatalyst in irradiation with energy; and
a cell adhesiveness changing pattern forming process of irradiating the cell adhesiveness changing material layer with energy to form a cell adhesiveness changing pattern changed adhesiveness of cells of the cell adhesiveness changing material layer.

8. A method of producing a cell culture base material comprising:
a pattern forming body base material forming process of forming a pattern forming body base material having a base material and a cell adhesiveness changing material layer formed on the base material and containing a cell adhesiveness changing material changing of adhesiveness of cells by the action of a photocatalyst in irradiation with energy; and
a cell adhesiveness changing pattern forming process of placing the pattern forming body base material and a . photocatalyst-containing layer-side base plate having a substrate and a photocatalyst-containing layer containing a photocatalyst so that the cell adhesiveness changing material layer and the photocatalyst-containing layer face, then, irradiating with energy from given direction to form a cell adhesiveness changing pattern changed adhesiveness of cells of the cell adhesiveness changing material layer.
